(19) United States
(12) Patent Application Publication
(10) Pub. No.: US 2014/0170655 A1
(43) Pub. Date: Jun. 19, 2014

(54) ISOLATION OF SELECTED MARKER-FREE MICOORGANISMS WITH A KNOWN GENETIC ELEMENT

(71) Applicants: Marie Just Mikkelsen, Bronshoj (DK); Thomas Kvist, Svogerslev (DK); Torbjørn Ølshøj Jensen, Stenløse (DK)

(72) Inventors: Marie Just Mikkelsen, Bronshoj (DK); Thomas Kvist, Svogerslev (DK); Torbjørn Ølshøj Jensen, Stenløse (DK)

(73) Assignee: BIOGASOL IPR APS, Ballerup (DK)

(21) Appl. No.: 14/126,344

(22) PCT Filed: Jun. 22, 2012
(86) PCT No.: PCT/EP2012/062150

§ 371 Date: Feb. 28, 2014

(30) Foreign Application Priority Data
Jun. 24, 2011 (EP) 11171328.5

(51) Int. Cl. C12Q 1/68

(52) U.S. Cl. CPC C12Q 1/689
USPC 435/6.11; 435/6.15; 435/6.12

(57) ABSTRACT

The invention relates to a method for isolating a microorganism containing a known genetic element. The method employs several rounds of 1) dilution of a mixed culture containing the selected microorganism in several replicates, 2) growing the replicates, 3) detecting the organism in at least one of the replicates and repeating steps 1) through 3) until the organism can be isolated by standard procedures.

### TECHNICAL FIELD

The invention relates to a method for isolating a microorganism containing a known genetic element, this organism being e.g. an antibiotic marker-free mutant bacterial cell or an organism in a screening study containing a desired gene fragment.

### BACKGROUND OF THE INVENTION

### Marker Free Deletion of Genes

Antibiotic resistance or other selectable marker genes are routinely used to select for the chromosomal insertion of heterologous genes or the deletion of native genes by homologous recombination to create new strains of Bacteria. The presence of antibiotic resistance genes in the host chromosome reduces the variety of plasmids that can be propagated in a cell, since these often rely on the same genes for their selection and maintenance. Genetically modified Bacteria containing chromosomal antibiotic resistance genes are undesirable for biological production, because the chromosomal DNA will be present in the final product as a low-level contaminant, with the risk of antibiotic resistance gene transfer to pathogenic Bacteria in humans or the environment. The insertion of a constitutively expressed marker gene can also alter the expression of adjacent chromosomal genes. Therefore, a rapid method of inserting genes into or deleting genes from bacterial chromosomes, resulting in strains with no antibiotic resistance genes or other selectable markers genes is a significant advantage [1].

One strategy for unlabeled (i.e., without a selectable marker gene) chromosomal gene integration relies on inserting a plasmid via a single homologous recombination event, followed by the removal of the plasmid by a second recombination event (resolution) to hopefully produce the desired genotype [1-3]. A major disadvantage of this approach is that if the insertion or deletion reduces the fitness of the cell, the resolution event will predominantly regenerate the wild-type rather than the mutant genotype and therefore can be inefficient.

An alternative method is to insert an antibiotic resistance gene flanked by regions of chromosomal homology, where recognition sites for a site-specific recombinase (SSR) immediately flank the antibiotic resistance gene. Chromosomal integration strategies include traditional RecA-mediated homologous recombination and recombineering using PCR products and phage-encoded recombination functions, including ET cloning that utilizes RecE/RecT from bacteriophage Rac or bacteriophage λ Red recombination [4]. Examples of SSRs/target sites used for antibiotic gene excision include Cre/loxP from bacteriophage P1 [5], Xer(Rip)/cis from Escherichia coli and Bacillus subtilis [1,6], Xis/attP from bacteriophage λ [7], and FLP/FRT [8] and R/RS [9] from the yeasts Saccharomyces cerevisiae and Zygosaccharomyces rouxii, respectively. The recombination functions of transposons such as Tn4430 from Bacillus thuringiensis [10] and thermostable rolling circle plasmids in Bacillus amyloliquefaciens [11] can also be employed.

The use of these systems is dependent on the functionality of these factors in the organism to be modified. For some organisms, no such system will work either due to high or low temperature optimum, requirements for high or low pH, and extreme concentrations of salts or other factors that will prevent the functionality of the recombinases. When using transposons or plasmids, such elements need to be functional in the organism of interest. Since in many organisms such elements have not been identified there remains a need for other methods for detecting gene insertion events. An alternative strategy is to use the sensitivity of microorganisms towards halogenated compounds such as haloacetate or 5-flouro-orotic acid. 5-flouro-orotic acid can be used to counter-select for the presence of the pyrF gene, since the product of pyrF converts 5-fluoro-orotic acid into the toxic compound 5-fluorouracil. Once the pyrF gene is deleted from the chromosome of the target microorganism, the pyrF gene can be used as a selection marker either on a plasmid or on a chromosomal integration in a different position. The method is widely used in yeast and has also been demonstrated in Clostridium thermocellum [12]. For this method to be effective, the organism of choice needs to be sensitive to halogenated compounds. Some industrial microorganisms including Thermoanaerobacter mathranii BG1 [13] are highly tolerant to toxic compounds such as halogenated compounds, and the use of these is therefore not possible. In general, microorganisms may become less sensitive to the toxic effects of halogenated compounds either by using novel pathways that circumvent the generation of reactive intermediates or by producing modified enzymes that decrease the toxicity of such compounds [14]. Also, once the pyrF gene has been used, it has to be removed before it can be used again as selection marker for a secondary mutation.

In plants, the removal of antibiotic resistance markers is also highly important. There are several ways to either avoid or get rid of selectable marker genes. Methods that will allow the removal of DNA in plants as efficiently as it is inserted have been developed, such as the use of site-specific recombination, transposition and homologous recombination. Researchers have also described several substitute marker genes that have no harmful biological activities. The presence of these non-bacterial genes allows the plants to metabolize non-toxic agents normally harmful to them [15].

### Isolation of Strains Containing a Gene Encoding a Commercially Important Product Such as an Enzyme

Currently, microorganisms are the major source for industrial enzymes in the feed sector [16]. Isolation of cells producing commercially important enzymes is a tedious process involving the screening of a great number of microbial cells before the right one is found. The screening can be based on a known DNA sequence, for instance a conserved motif in the enzyme, or it can be based on the detection of the enzyme's activity in the culture supernatants. The source material can be plant or animal matter, or microbes—both prokaryotes (e.g. Bacteria and Archaea) and eukaryotes (e.g. Yeast and Fungi) [16].

One problem often encountered early in the screening process is that natural microbial isolates usually produce commercially important enzymes in exceedingly low concentrations. In some cases screening methods based on the activity of the enzyme is therefore not successful [16].

Other commonly known screening methods includes colony hybridization, PCR or enzyme assays. In either case, the cells are isolated as colonies on plates or as pure liquid cultures before they are screened. The screening process involves the examination of thousands of samples of soil, plant material, etc., and the random isolation and screening of the resident microbial flora. Although capable of significant automation, the throughput capacity largely determines the speed of progress. [16].

An alternative approach is to clone the gene of interest directly from the mixed cell population into a host cell. However, it may not be possible to express the product in the foreign host and the efforts may therefore be futile [16].

In the last 50 years, over half of the major breakthroughs in the pharmaceutical industry have been natural products. Today, 60% of chemotherapeutics entering late stage clinical trials are of microbial origin. Methods of identifying and isolating microorganisms producing pharmaceuticals, biochemicals or chemical building blocks are largely the same as those described for isolation of enzyme producing strains. Thus there exists a need for improved methods for isolating specific microbial cells from a cell population, where the method does not rely on the detection of a specific metabolic or enzyme product, and which is efficient.

The present invention provides a method for isolating a microorganism containing a known genetic element, this organism being e.g. an antibiotic marker-free mutant bacterial cell or an organism in a screening study containing a desired gene fragment.

### SUMMARY OF THE INVENTION

The present invention pertains to a technique in which the frequency of the organism containing the DNA fragment of interest is increased stepwise, by several rounds of 1) dilution of a culture containing the selected microorganism in several replicates, 2) growing the replicates, 3) detecting the organism in at least one of the replicates and repeating steps 1) through 3) until the organism can be isolated by standard procedures.

The invention is based on the concept that if a selected microorganism, present in a diluted microbial population, is found at a concentration where the probability of finding it is less than 1, the frequency of the selected microorganism will be higher than in the less diluted population. For each round of selection, the frequency of the organism of interest increases until it can be isolated by standard methods such as plating and detection of the DNA fragment in the isolated organisms by PCR, hybridization or other assays.

When using the method of the invention, screening experiments can, to a surprising degree, be reduced from screening of thousands of isolated cells to screening of a few hundred.

The present invention provides a method for isolating a selected microorganism from a mixed culture of microorganisms comprising the steps:
a) providing a mixed culture of microorganisms containing said selected microorganism, wherein said selected microorganism comprises one or more nucleic acid molecule, wherein said nucleic acid molecule comprises a known unique consecutive sequence of at least 15 nucleic acid base pairs, and wherein the frequency of the selected microorganism is less than 10⁻³,
b) serially diluting said mixed culture in a growth medium to provide diluted cultures;
c) incubating said diluted cultures to allow growth of said microorganisms;
d) detecting the presence or absence of said nucleic acid molecule in said diluted cultures obtained from step (c) to allow the frequency of said selected microorganism in said mixed culture to be determined, and identifying the most dilute culture in which said nucleic acid molecule is detected (P), and identifying the least diluted culture in which said nucleic acid molecule is not detected (N), wherein the dilution factor between P and N is D and the total dilution factor of culture N relative to the undiluted mixed culture is Dt;
e) preparing and incubating replicate diluted cultures having the dilution Dt;
f) detecting the presence or absence of said nucleic acid molecule in replicate dilution cultures obtained from step (e), wherein the frequency of said selected microorganism in said replicate dilution cultures is increased compared to culture P;
g) selecting a replicate dilution culture containing said nucleic acid molecule and using said selected culture to repeat steps (e) to (g), wherein the total dilution factor (Dt) of the replicate diluted cultures is increased by the factor D, and wherein steps (e) to (g) are repeated until the frequency of said selected microorganism comprising said nucleic acid molecule is greater than 10⁻³, preferably greater than 10⁻¹;
h) screening single colonies of a replicate dilution culture obtained from step (g) and isolating said selected microorganism comprising said nucleic acid molecule.

### LEGENDS TO FIGURES

FIG. 1. A graphical representation of the delivery vector used to introduce the genetic fragment into the genome of the organism. The PAR fragment (parM-ext) is placed between the up flank (LDH-Up) and the down flank (LDH-Down) in the clockwise orientation. Ndel shows the site where the vector is linearized prior to transformation. parM-ext-Re (100%) and parM-ext-fw3 (100%) are priming sites used for screening. Features illustrated between LDH-Down and LDH-Up clockwise orientation originate from cloning vector pUC19.

FIG. 2. An agarose gel showing screening result of the 10⁻⁵ mixtures (samples 5Am-5Dm) and associated negative and positive controls. Square box shows selected sample 5Cm, chosen for individual sample differentiation).

FIG. 3. An agarose gel showing the PCR products from the individual samples of mixture 5Cm (FIG. 2). 5C1 was found to be the one sample with parM-ext inserted in the genome.

FIG. 4. An agarose gel showing PCR products from the individual samples from mixture 6Dm (10⁻⁶). 6D4 was found to be one of four samples with parM-ext inserted in the genome.

FIG. 5. An agarose gel showing PCR products from individual samples of mixture 7Dm (10⁻⁷). 7D5 was found to be the sample with parM-ext inserted in the genome.

FIG. 6. An agarose gel showing PCR results from individual samples of mixture 8Am (10⁻⁸). 8A5 was found to be the sample with parM-ext inserted in the genome.

FIG. 7. An agarose gel showing PCR results from individual samples of mixture 9Am (10⁻⁹). 9A2 was found to be one of two samples with parM-ext inserted in the genome.

### DETAILED DESCRIPTION OF THE INVENTION

The present invention pertains to a method for isolating a selected microorganism from a mixed culture of microorganisms without the need for marker based selection techniques, such as antibiotic resistance marker genes.

A mixed culture of microorganisms is a population of microorganisms where the individual microorganisms within the population differ with respect to a known consecutive sequence of at least 15 nucleic acid base pairs in their DNA (e.g. chromosomal or plasmid DNA molecules). The population of microorganisms in the mixed culture comprises a selected microorganism.

A selected microorganism is a specific microorganism present within the mixed culture of microorganisms, wherein cells of the specific microorganism comprise one or more nucleic acid molecule (e.g. chromosomal or plasmid DNA), and wherein the molecule comprises a known consecutive sequence of at least 15 nucleic acid base pairs (or nucleotides) that are not present in cells of the other microorganisms in the culture. Cells of the specific microorganism can be selected from the mixed culture of microorganisms, by selecting for a microorganism cell comprising this at least 15 consecutive nucleic acid base pairs. The specific microorganism can also be selected from the mixed culture of microorganisms, by selecting for a microorganism cell comprising at least two nucleic acid molecules comprising at least 15 nucleic base pairs, and wherein the at least two molecules are comprised within a larger nucleic acid molecule comprising 50 to 10,000 nucleic acid base pairs, preferably 150 to 3,000 nucleic acid base pairs and even more preferably 150 to 1500 nucleic acid base pairs.

The isolation of the selected microorganism present in a mixed culture of microorganisms using the method of the invention is particularly suitable where the frequency of the selected microorganism in the mixed culture is less than 10⁻³. The method of the invention is also suitable where the frequency of the selected microorganism in the mixed culture is 10⁻⁴, 10⁻⁵, 10⁻⁶, 10⁻⁷ or lower.

This method for isolating a selected microorganism employs a surprisingly effective technique that is schematically represented in Table 1. In Table 1 each tube illustrates a container, wherein the microorganism is grown. This container could also be a well in a microtitre plate or any other enclosed space containing a liquid growth medium.

Growth medium, in liquid form, is used to culture the mixed culture of microorganisms and dilutions of this culture. The growth medium serves to support growth of the microorganisms, and the composition of the medium is adapted to provide all essential nutrients required for the growth of the respective microorganism. The method does not rely on, nor requires, that the growth medium selectively promotes the growth of the specific microorganism to be selected, and hence can be a non-selective growth medium.

In (a) a tube is shown comprising a mixed culture of microorganisms, wherein the culture comprises a selected microorganism.

In step (b) the mixed culture is diluted by consecutive transfer into liquid growth medium. The number of dilutions will depend on the cell density of the mixed culture, but typically dilutions ranging from 10⁻²-10⁻⁹ are contemplated; more typically a dilution range extending down to 10⁻⁶ is suitable. The dilution factor is preferably 1:10, although smaller or greater dilution factors are contemplated. It is both contemplated and sufficient that each dilution of the mixed culture is represented by one dilution culture.

The diluted cultures in step (b) are then incubated until sufficient cell mass is obtained for the subsequent detection step. The incubation conditions employed to support growth of the microorganism are adapted to meet the growth requirements of the respective microorganism. Selection of growth temperature, supply of air for aerobic growth, or non-aerobic growth conditions, shaking conditions are all optimized to support growth based on the known growth requirements of the respective microorganism. The period of incubation is selected based on the growth rate of the respective microorganism, but will normally continue until the growth medium no longer provides conditions suitable for growth of the organism e.g. if the growth medium nutrients are exhausted.

The detection step is used to detect the presence of the one or more nucleic acid molecule, that comprises a known consecutive sequence of at least 15 nucleic acid base pairs, and that is unique to the selected microorganism in one or more of the dilutions of the mixed culture. It is contemplated that the detection step is performed on at least two, and preferably more than two of the dilutions of the mixed culture prepared and cultivated in step (b), where each dilution to be screened is represented by one dilution culture. The nucleic acid detection method will normally be optimized for the respective microorganism. Release of nucleic acid molecules (DNA) from a cell for the purpose of DNA detection normally requires cell disruption or cell permeabilisation. Preferably total DNA is extracted from a sample of each dilution culture. Methods for detection of the one or more nucleic acid molecule that is unique to the selected microorganism include Polymerase Chain Reaction (PCR) employing nucleic acid primers that can specifically amplify the one or more nucleic acid molecule. Other methods of nucleic acid molecule detection include hybridization with DNA probes that hybridize specifically with the nucleic acid molecule or its complementary strand. Suitable methods for DNA extraction and DNA detection by PCR or hybridization are detailed in standard textbooks e.g. Molecular Cloning a laboratory manual [17] The DNA detection step serves to identify which dilutions comprise the selected microorganism.

The frequency of said selected microorganism in said mixed culture is determined by dividing the dilution of the dilution culture where the said microorganism can be detected by the dilution of the most dilute sample in the dilution series where growth of the mixed microbial culture is observed.

The culture originating from the most diluted sample of the mixed culture in which the one or more nucleic acid molecule unique to the selected microorganism is detected is named P. The culture originating from the least diluted sample of the mixed culture in which the one or more nucleic acid molecule unique to the selected microorganism can no longer be detected is named N. The dilution factor between the diluted samples from which P and N originate is named D, where D is greater than 1, and will preferably be 10; and the total dilution factor for obtaining culture N relative to the undiluted mixed culture is Dt.

In step (c) between 2 and 500 replications of dilution culture N (starting from P and having the dilution factor Dt) are made in growth medium in order to increase the probability of finding the selected microorganism in at least one of these replicates. If dilution culture N originates from a 10-fold dilution of the diluted sample from which P originates, at least 10-20 replicates will be made of dilution culture N. The replicates of dilution culture N are allowed to grow in step (d) until sufficient cell mass is obtained for the subsequent detection step (e). Typically, the replicates will be grown under the same conditions as in step (b) to give approximately the same cell density as the cell culture from which the dilution was made (P). The presence of the one or more nucleic acid molecule that is unique to the selected microorganism is then detected in the replicate cultures of N, step (e). Only a fraction of the replicate dilution cultures of N will contain the nucleic acid molecule of the selected microorganism. However, in the cultures where the nucleic acid molecule of the selected microorganism is now detected, the frequency of the selected microorganism relative to other organisms in the culture will be higher. If for example the selected microorganism is detected in two out of 20 cultures, the frequency of the selected microorganism will now be approximately 10 fold higher than in the dilution culture (P) from which the dilutions were made. In step (f), a replicate dilution culture of N, in which the selected microorganism is detected, is then used as culture P for performing a new step (c) in a secondary cycle of dilution and selection. This cycle will be repeated until the frequency of the selected microorganism is greater than 10⁻³, preferably 10⁻² or even more preferably higher than 10⁻¹. The number of repetitions of steps (c) to (f) is generally at least 1, but is more likely to require 2, 3, 4, 5 or 6 or more repetitions, where after the selected microorganism can then be isolated from a replicate dilution culture (N) comprising the selected microorganism, by using standard techniques for single cell colony isolation such as plating on solid growth medium, incubation and growth of single cell colonies followed by detection of the one or more nucleic acid molecule of the selected microorganism.

The method of the invention has the major and surprising advantage that it reduces the number of screening experiments from screening of thousands of isolated cells to screening of a few hundred. The method can be used to isolate a selected microorganism characterized by the deletion of a nucleic acid sequence (e.g. gene deletion mutant) or by the insertion of a nucleic acid sequence (e.g. gene insertion mutant). The method can also be used to isolate a selected microorganism characterized by containing a natural unique nucleic acid sequence which is not present in other microorganisms in the mixed culture.

The method is suitable for any microorganism capable of single cell growth in liquid culture, in particular bacterial and fungal (e.g. yeast) cells capable of single cell growth. The method is particularly useful for making marker-free deletions or insertions in extremophiles such as:
The acidophilic Archaea Sulfolobales, Thermoplasmatales, ARMAN (Archaeal Richmond Mine Acidophilic Nanoorganisms), Acidianus brierleyi, A. infernus and Metallosphaera sedula, and the acidophilic Bacteria Acidobacterium and Acidithiobacillales, Thiobacillus prosperus, Thiobacillus acidophilus, Thiobacillus organovorus, Thiobacillus cuprinus and Acetobacter aceti.
The alkaliphilic Bacteria Geoalkalibacter ferrihydriticus, Bacillus okhensis, and Alkalibacterium iburiense
The halophilic Archaea belonging to the family of Halobacterium and halophilic bacterium Halobacterium halobium and Chromohalobacter beijerinckii.
The hyperthermophilic archea Methanopyrus kandleri, Pyrolobus fumarii, Pyrococcus furiosus, and the hyperthermophilic bacterium Geothermobacterium ferrireducens, and Aquifex aeolicus.
Thermophilic Bacteria belonging to the Bacillus stearothermophilus species and the Thermoanaerobacter genus.

The method is particularly useful for isolating microbial cells which produce a product for which no selection procedure exist e.g. cells producing chemical building blocks such as:
acids (such as maleic-, aspartic-, malonic-, propionic-, succinic-, fumaric-, citric-, acetic-, glutamic-, itaconic-, levulinic-, acotinic-, glucaric-, gluconic-, and lactic-acid),
amino acids (such as serine, lysine, threonine),
alcohols (ethanol, butanol, propanediol, butanediol, arabitol) or
other high value products (such as acetoin, furfural, and levoglucosan), or
cells which produce an enzyme in amounts that is insufficient for a selection procedure.

The method is particularly useful for isolating microbial cells which are present in the mixed culture of microorganisms at a frequency which is insufficient for detection of the activity of an expressed microbial enzyme.

A method of the invention solves the problem of how to isolate a selected microorganism without the need for any form of marker gene for selection purposes, and without the need to introduce genes into the microorganism to be selected such as a complete gene, [18] a heterologous recombinase, an antibiotic resistance marker, a plasmid or a transposon into the selected microorganism.

### EXAMPLES

### Materials and Methods

The following materials and methods were applied in the Examples below:

Enzymes and reagents: If not stated otherwise enzymes were supplied by MBI Fermentas (Germany) and used according to the suppliers recommendations. PCR-conditions were (15sec/15sec/15sec)ₓ₂₅ at temperatures (94° C./60° C. /72° C.). The products were amplified in a Techne PCR machine with Fermentas DreamTaq polymerase in PCR buffer (160 mM (NH₄)₂SO₄; 670 mM Tris-HCl (pH 8.8); 0.1% Tween-80, 1 mM Cresol Red, 0.125, 0.125M Ficoll 400).

Gel electrophoresis: PCR results were evaluated on the basis of agarose gel electrophoresis using BioRad SubCell Equipment. Gels were run at 80V in 1% agarose for 20-30 minutes. Visualization was done by casting Ethidium Bromide (0.5 μg/ml) into the gels

Roll tube isolation: Hungate roll tubes [19] were used to isolate axenic cultures from solid surface cultivations. Isolations were transferred to liquid BA media [20].

Matrix screening of multiple samples: To establish a system for screening multiple samples simultaneously, pooling of samples was used. 20 samples were arranged in a matrix of 4 columns (A-D)×5 rows (1-5). 400 μl from each sample (each column, A through D) were pooled into a single tube, and DNA from the mixture was used as PCR template. Individual samples from a column resulting in positive PCR were extracted and PCR amplified.

### Example 1

### Marker-Free Gene Deletion and Insertion in a Thermoanaerobacter Strain

### 1.1 Strains and Growth Conditions

Thermoanaerobacter strain BG10 is deposited with DSMZ (DSMZ—Deutsche Sammlung von Mikroorganismen and Zellkulturen GmbH, Mascheroder Weg 1b, 38124 Braunschweig) under deposit number 23015. All microbial strains were cultured at 70° C. anaerobically in minimal medium (BA) with 2 g/L yeast extract as in unless otherwise stated. For solid medium, roll tubes containing BA medium with 11 g/L phytagel and additional 3.8 g/L MgCl₂.6H₂O was used., Escherichia coli Top10 (Invitrogen, USA) was used for cloning purposes. Top10 was routinely cultivated at 37° C. in Luria-Bertani medium [17] supplemented with 100 μg/mL ampicillin when needed.

### 1.2 Construction of Thermoanaerobacter BG10Δldh Strain

The LDH gene was deleted from the Thermoanaerobacter BG10 wt strain (DSM 23015) by homologuous recombination as described in [13] to generate LDH deficient strain BG10XL.

1.3 Construction of the parM-ext Insertion Cassette

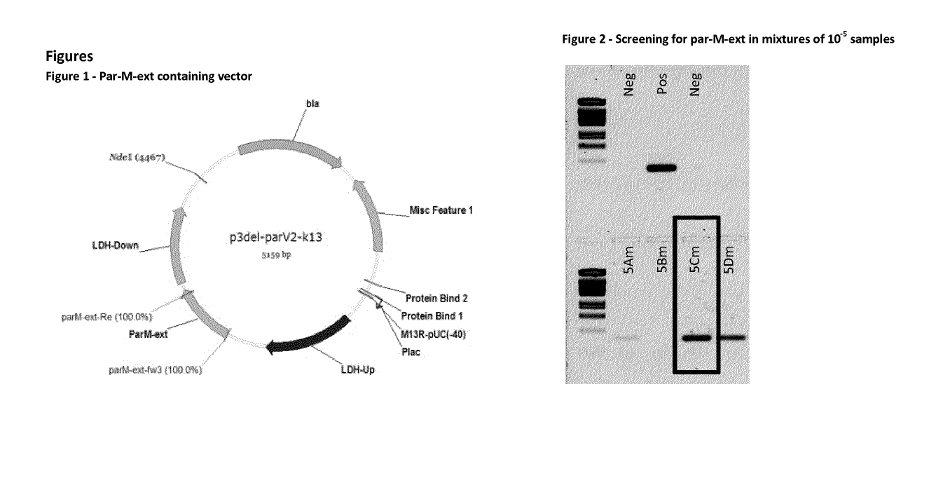

The DNA fragment used for insertion of the parM-Ext fragment into the lactate dehydrogenase region of BG10XL genome was cloned in the vector p3del-ParV2-K13, shown in FIG. 1, and contains:
1) a DNA fragment upstream of the l-ldh gene of BG10, amplified using primers

Idhup1F
[SEQ ID NO: 1]
(5′-TTC CAT ATC TGT AAG TCC CGC TAA AG-3′);
and
Idhup2R
[SEQ ID NO: 2]
(5′-ATT AAT ACA ATA GTT TTG ACA AAT CC-3′),

2) A non-coding parM-ext fragment used solely for identification, amplified using

primers:
parM-ext-Fw
[SEQ ID NO: 3]
(5′-CCC CCC GTT AAC ATC AAA CTA CAG
TGG CAG GAA AG-3′);
&
parM-ext-re
[SEQ ID NO: 4]
(5′-CCC CCC TGC AGC GTT
GCT TCA GAT AGT TAT TAT CTT TTC TG-3');

3) a DNA fragment downstream of the l-ldh gene of BG10, amplified using primers

Idhdown3F
[SEQ ID NO: 5]
(5′-ATA TAA AAA GTC ACA GTG TGA A-3′);
and
Idhdown4R
[SEQ ID NO: 6]
(5′-CAC CTA TTT TGC ACT TTT TTT C-3′).

The p3del-ParV2-k13 vector was amplified in E. coli GM2163 (CGSC 6581) and isolated using midi-size preparation (Nucleobond) as described in [21].

1.4 Linearization of the Vector - p3del-ParV2-K13 Comprising the parM-ext Insertion Cassette

Quantification of the vector prior to digestion was carried out using an Eppendorf BioPhotometer, using the built in “DNA ds quantification”. 50 μl vector p3del-ParV2-K13 was digested using restriction enzyme Ndel in a 100 μl reaction using 5 μl Ndel fast Digest Enzyme. Digestion was carried out over night (ON) at 37° C. in Fermentas fast digest buffer. Linearization was verified on a 0.7% agarose gel.

1.5 Transformation of Thermoanaerobacter BG10XL with Linearized p3del-parM-ext

100 μl Ndel digested vector (˜10 μg/μl) (p3del-parM-ext) was cooled to 0° C. and mixed with 100 μl fully grown culture Thermoanaerobacter BG10XL. The mixture was transferred to pre-cooled growth media, and incubated at 70° C. for 16 hours. Subsequent to transformation and 16 hours incubation, four consecutive cultivation steps were applied using an inoculum of 1%. Consecutive transfers were implemented to eliminate a false positive signal originating from the Ndel digested transformation template rather than from the actual targeted inserted fragment.

1.6 PCR Detection of parM-ext Fragment

PCR-conditions applied in the screening procedure were: Denaturing at 94° C. (15 s), annealing at 60° C. (15 s), elongation at 72° C. (15 s). The PCR cycle was repeated 25 times in a Techne Progene PCR machine. Polymerase used was DreamTaq polymerase (Fermentas) and primers used in the screening were; parM-ext-Fw3 ([SEQ ID NO:7] 5′-GGC AAT ACA GCG ACG TTA ATG-3′) parM-ext-Re ([SEQ ID NO:8] 5′-CCC CCC TGC AGC GTT GCT TCA GAT AGT TAT TAT CTT TTC TG-3′).

1.7 Detection and Isolation of Marker Free Thermoanaerobacter BG10XL Transformed with parM-ext Insertion Cassette

The following steps were then performed to isolated and identify a Thermoanaerobacter BG10XL, transformed with parM-ext insertion cassette. Starting from the fully grown culture obtained immediately after the fourth transfer had successfully been completed, an initial 10-fold dilution series were set up in growth medium and incubated under the same growth conditions (Table 2).

Growth was detected in cultures diluted up to 10⁻⁹. DNA was isolated from each of the cultures, followed by PCR screening for the presence of the parM-ext fragment. The fragment was only detected in dilutions from 10⁻¹ to 10⁻⁴. 20 replicates of 10⁻⁵ dilutions of the same culture (where the parM-ext fragment was not detectable) were prepared, and incubated over night to obtain fully grown cultures.

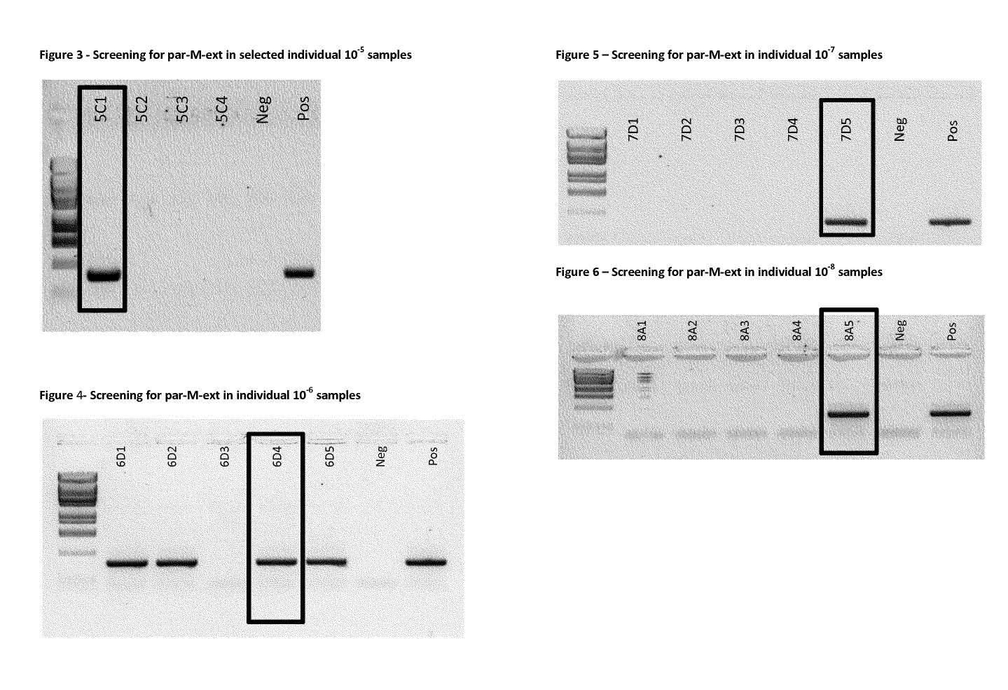

Four mixtures, each containing five individual dilution replicates (in total 20) were analyzed by PCR for the presence of the PAR-M-ext sequence (FIG. 2). As seen FIG. 2, at least two out of four mixtures (composed of pooled 10⁻⁵ dilution cultures) contained parM-ext (5Cm and 5Dm). Further analysis of the individual five cultures from mixture 5Dm revealed that one out of four individual cultures in the 5Cm mixture contained the inserted parM-ext sequence (5C1, FIG. 3).

The fully grown culture 5C1 was used to make 20 dilution cultures each diluted by an additional factor of 10 fold to give a total dilution of 10⁻⁶ with respect to the undiluted starting culture (5C1). The cultures were then grown overnight. Again, the presence of the parM-ext fragment was analyzed in mixtures of pooled samples of the 10⁻⁶ dilution cultures and was subsequently identified in the single cultures 6D1, 6D2 and 6D4 (FIG. 4).

Tube 6D4 was used to set up 20 dilution cultures, each diluted by an additional factor of 10 fold to give a total dilution of 10⁻⁷ with respect to the undiluted starting culture (6D4), and the cultures were allowed to grow overnight. The presence of the parM-Ext fragment was identified in culture 7D5 as seen in FIG. 5.

7D5 was used to make 20 dilution cultures, each diluted by an additional factor of 10 fold to give a total dilution of 10⁻⁸ fold with respect to the undiluted starting culture (7D5), which were allowed to grow over night. The presence of the parM-ext fragment was identified in culture 8A5 as seen in FIG. 6.

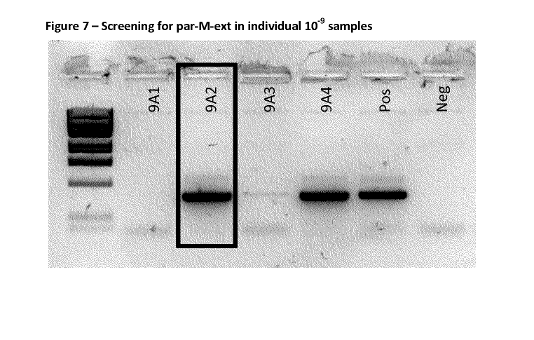

8A5 was used to make 20 dilution cultures, each diluted by an additional factor of 10 fold to give a total dilution of 10⁻⁹ fold with respect to the undiluted starting culture (8A5), which were allowed to grow. The presence of the parM-ext fragment was identified in culture 9A2 as seen in FIG. 7.

From 9A2, roll tubes were prepared in order to isolate pure cultures with a parM-ext genomic insertion. After two days of incubation, 5 single colonies were picked from Hungate Roll Tubes and incubated in each 10 ml of liquid medium. Two out of five monocultures contained the parM-ext fragment. The correct insertion of the parM-ext fragment [SEQ ID NO:9] was verified using primers in regions upstream and downstream of the lactate dehydrogenase.

The resulting PCR positive cultures were checked by PCR using primers annealing outside the region used for homologous recombination. In this way, ldh loci in which no recombination have taken place will also be amplified although the fragment will be of different length. Primers LDH-out-Up ([SEQ ID NO:10] 5′-GAG CTG CTT TAA GTG TCT CAG G-3′) and LDH-out-Dn3 ([SEQ ID NO:11] 5′-GAA GTG GAT CCT TTA TAG GCC GGT-3′). PCR conditions were identical to those described under “PCR detection of parM-ext fragment” but with a prolonged elongation time (2 minutes 30 seconds).

The lactate dehydrogenase was efficiently removed and replaced with a parM-ext fragment without the need for an antibiotic resistance gene or any other functional DNA sequence to be incorporated into the genome of the bacterium. A total of 150 cultures and PCR reactions were used to find the selected organism which has a frequency of 10⁻⁴. Should the identification have been made without the use of the current invention, which serves to increase the frequency of the selected microorganism, at least 10,000 single cultures would have had to be grown and PCR analyzed.

The resulting strain is deposited in the German Resource Centre for Biological Material (DSMZ) under the name Thermoanaerobacter italicus Pentocrobe 3100-401 with deposition number DSM 24725.

### Example 2

### Isolation of a Potential 2,3-butanediol Producing Bacterial Strain from Cow Manure

### 2.1 Growth Conditions

Growth medium comprising reduced LB (50% Yeast extract and 50% Tryptone) [17], supplemented with biomass derived C5 sugar (25 g/l xylose), was inoculated with cow manure. After inoculation, the culture was kept free of atmospheric air. Incubation was performed at a constant temperature at 37° C. with shaking 175 rpm. For cultivation on solid medium, technical grade xylose (25 g/l ) was added to the reduced LB medium supplemented with 15 g/l agar.

2.2 Screening for a Potential 2,3-butanediol Producer

The enzyme, 2,3-butanediol dehydrogenase (EC 1.1.1.4), is used solely in the production of 2,3-butanediol. PCR, using the primers budC_det_{—}247_forward ([SEQ ID NO: 12] 5′-AAC GTS ATT GTG AAT AAC GCM GG-3′) and budC_det_{—}684_reverse ([SEQ ID NO:13] 5′-ATC TTC CGG CTC NGA NAG GC-3′) were used to detect the presence the budC gene [SEQ ID NO:14], which encodes 2,3-butanediol dehydrogenase. PCR conditions were identical to those described under “PCR detection of parM-ext fragment”.

After the mixed culture (derived from an environmental cultivation) was fully grown, a series of 10-fold dilutions of the culture were generated by consecutive transfers into growth media. The dilutions were incubated under the same condition (as described in “Growth conditions”) to obtain fully grown cultures.

DNA was isolated from each of the individual dilution cultures. The presence of the 2,3-butanediol dehydrogenase fragment was detected by PCR using budC_det_{—}247_forward and budC_det_{—}684_reverse, producing an amplified fragment of 438 base pairs.

Growth was detected in cultures diluted up to 10⁻⁹, while the budC-fragment was detected (by PCR screening) in dilutions from 10⁻¹ to 10⁻⁵. 20 replicates of 10⁻⁶ dilutions of the same culture were prepared, and incubated until the cultures were fully grown.

PCR screening of the 20 replicates of the 10⁻⁶ dilution cultures was performed using a screening matrix as described in “matrix screening of multiple samples”. The three mixtures 6Am, 6Bm, and 6Cm were PCR positive for the presence of budC-fragment, whereas 6Dm was negative. Further analysis of the individual five cultures of 6Am revealed that one 6A1 contained the budC-fragment.

The fully grown culture of 6A1 was then used to prepare 20 dilution cultures each diluted by an additional factor of 10 fold to give a total dilution of 10⁻⁷ fold with respect to the undiluted starting culture (6A1). The cultures were then incubated until fully grown. The presence of budC-fragment was analyzed among the pooled samples from the cultures. The presence of budC-fragment was detected in four of the 20 cultures including 7C4.

Tube 7C4 was used to make 20 dilutions cultures each diluted by an additional factor of 10 fold to give a total dilution of 10⁻⁸ fold with respect to the undiluted starting culture (7C4), which were incubated until fully grown. The presence of the budC-fragment was identified in 8D3.

8D3 was used to make 20 dilution cultures each diluted by an additional factor of 10 fold to give a total dilution of 10⁻⁹ fold with respect to the undiluted starting culture (8D3), which were incubated until fully grown. The budC-fragment was detected in 9A5 and in two others, of the 20 cultures.

From 9A5, cultivation plates were prepared according the media in “Growth conditions”. When colonies appeared on the plates 25 single colonies were picked. Colony-PCR was performed by suspension of the picked colony in 20 μl NucleotideFree water. One μl of each suspended colony served as template for the PCR with the above used budC-detection primers. In total, the presence of the budC-fragment was detected in six of the 25 single colonies.

### REFERENCES

1 Bloor, A. E. & Cranenburgh, R. M. An efficient method of selectable marker gene excision by Xer recombination for gene replacement in bacterial chromosomes. Appl Environ Microbiol 72, 2520-2525, (2006).
2 Leenhouts, K. et al. A general system for generating unlabelled gene replacements in bacterial chromosomes. Mol Gen Genet 253, 217-224, (1996).
3 Link, A. J., Phillips, D. & Church, G. M. Methods for generating precise deletions and insertions in the genome of wild-type Escherichia coli: application to open reading frame characterization. J Bacteriol 179, 6228-6237, (1997).
4 Court, D. L., Sawitzke, J. A. & Thomason, L. C. Genetic engineering using homologous recombination. Annual Review of Genetics 36, 361-388, (2002).
5 Leslie, N. R. & Sherratt, D. J. Site-specific recombination in the replication terminus region of Escherichia coli: functional replacement of dif. Embo Journal 14, 1561-1570, (1995).
6 Morimoto, T., Ara, K., Ozaki, K. & Gasawara, N. A new simple method to introduce marker-free deletions in the Bacillus subtilis genome. Genes & Genetic Systems 84, 315-318, (2009).
7 Zubko, E., Scutt, C. & Meyer, P. Intrachromosomal recombination between attP regions as a tool to remove selectable marker genes from tobacco transgenes. Nature Biotechnology 18, 442-445, (2000).
8 Datsenko, K. A. & Wanner, B. L. One-step inactivation of chromosomal genes in Escherichia coli K-12 using PCR products. Proceedings of the National Academy of Sciences of the United States of America 97, 6640-6645, (2000).
9 Sugita, K., Kasahara, T., Matsunaga, E. & Ebinuma, H. A transformation vector for the production of marker-free transgenic plants containing a single copy transgene at high frequency. Plant Journal 22, 461-469, (2000).
10 Sanchis, V., Agaisse, H., Chaufaux, J. & Lereclus, D. A recombinase-mediated system for elimination of antibiotic resistance gene markers from genetically engineered Bacillus thuringiensis strains. Applied and Environmental Microbiology 63, 779-784, (1997).
11 Zakataeva, N. P., Nikitina, O. V., Gronskiy, S. V., Romanenkov, D. V. & Livshits, V. A. A simple method to introduce marker-free genetic modifications into the chromosome of naturally nontransformable Bacillus amyloliquefaciens strains. Applied Microbiology and Biotechnology 85, 1201-1209, (2010).
12 Tripathi, S. A. et al. Development of pyrF-Based Genetic System for Targeted Gene Deletion in Clostridium thermocellum and Creation of a pta Mutant. Applied and Environmental Microbiology 76, 6591-6599, (2010).
13 Mikkelsen, M., Just (Stenløpsevej 52, Brønshøj, DK-2700, DK) & Ahring, B., Kiær (Fruerhøj 43, Hørsholm, DK-2970, DK). Thermoanaerobacter mathranii Strain BG1. (2007).
14 Vlieg, J., Poelarends, G. J., Mars, A. E. & Janssen, D. B. Detoxification of reactive intermediates during microbial metabolism of halogenated compounds. Current Opinion in Microbiology 3, 257-262, (2000).
15 The International Service for the Acquisition of Agri-biotech Applications (ISAAA). Pocket K No. 36: Marker-Free GM Plants. (2009).
16 Lorenz, P. & Eck, J. Metagenomics and industrial applications. Nat Rev Micro 3, 510-516, (2005).
17 Sambrook, J. & Russell, D. W. Molecular Cloning a laboratory manual. (Cold Spring Harbor Laboratory Press, 2001).
18 Ethanol production by thermophilic bacteria: physiological comparison of solvent effects on parent and alcohol-tolerant strains of clostridium thermohydrosulfuricum. Applied And Enviromental Microbiology 48, 171-177, (1984).
19 Hungate, R. E. in Methods in microbiology eds J. R. Norris & D. W. Ribbons) Ch. 4, 118-132 (Academic Press, 1969).
20 Angelidaki, I., Petersen, S. P. & Ahring, B. K. Effects of Lipids on Thermophilic Anaerobic-Digestion and Reduction of Lipid Inhibition Upon Addition of Bentonite. Applied Microbiology and Biotechnology 33, 469-472, (1990).
21 Mikkelsen, M., Just (Stenmaglevej 13, Brønshøj, DK-2700, DK) & Yao, S. G. P., st. th, Søborg, DK-2860, DK). Increased Ethanol Production in Recombinant Bacteria. (2010).

1. A method for isolating a selected microorganism from a mixed culture of microorganisms comprising the steps:
a) providing a mixed culture of microorganisms containing said selected microorganism, wherein said selected microorganism comprises one or more nucleic acid molecule, wherein said nucleic acid molecule comprises a known unique consecutive sequence of at least 15 nucleic acid base pairs, and wherein the frequency of the selected microorganism is less than 10-3,
b) serially diluting said mixed culture in a growth medium to provide diluted cultures;
c) incubating said diluted cultures to allow growth of said microorganisms;
d) detecting the presence or absence of said nucleic acid molecule in said diluted cultures obtained from step (c) to allow the frequency of said selected microorganism in said mixed culture to be determined, and identifying the most dilute culture in which said nucleic acid molecule is detected (P), and identifying the least diluted culture in which said nucleic acid molecule is not detected (N), wherein the dilution factor between P and N is D and the total dilution factor of culture N relative to the undiluted mixed culture is Dt;
e) preparing and incubating replicate diluted cultures having the dilution Dt;
f) detecting the presence or absence of said nucleic acid molecule in replicate dilution cultures obtained from step (e), wherein the frequency of said selected microorganism in said replicate dilution cultures is increased compared to culture P;
g) selecting a replicate dilution culture containing said nucleic acid molecule and using said selected culture to repeat steps (e) to (g), wherein the total dilution factor (Dt) of the replicate diluted cultures is increased by the factor D, and wherein steps (e) to (g) are repeated until the frequency of said selected microorganism comprising said nucleic acid molecule is greater than 10-3, preferably greater than 10-1;
h) screening single colonies of a replicate dilution culture obtained from step (g) and isolating said selected microorganism comprising said nucleic acid molecule.

2. The method according to claim 1, wherein the frequency of the selected microorganism in step a) is between 10-4 and 10-7.

3. The method according to claim 1, wherein the selected microorganism is a deletion and/or insertion mutant of a microorganism.

4. The method according to claim 1, wherein the selected microorganism is a bacterial cell.

5. The method according to claim 4, wherein the bacterial cell belongs to the family of Thermoanaerobacteriaceae.

6. The method according to claim 4, wherein the bacterial cell belongs to the genus of Thermoanaerobacter.

7. The method according to claim 1, wherein said nucleic acid molecule encodes an enzyme.

8. The method according to claim 7, wherein said enzyme is selected among an oxidoreductase, transferase, hydrolase, lyase, isomerase and ligase.

9. The method according to claim 8, wherein said enzyme catalyses a metabolic step required for production of maleic acid, aspartic acid, malonic acid, propionic acid, succinic acid, fumaric acid, citric acid, acetic acid, glutamic acid, itaconic acid, levulinic acid, acotinic acid, glucaric acid, gluconic acid and lactic acid, amino acids, alcohol, acetoin, furfural, and levoglucosan.

10. The method according to claim 1, wherein said nucleic acid molecule is detected by PCR.

11. The method according to claim 1, wherein said nucleic acid molecule is detected by hybridization to said nucleic acid molecule.

12. The method according to claim 1, wherein said one or more nucleic acid molecule, comprises at least two nucleic acid molecules, wherein each of said two molecules comprises a known unique consecutive sequence of at least 15 nucleic acid base pairs, and wherein the at least two molecules are comprised within a larger nucleic acid molecule comprising 50 to 10,000 nucleic acid basepairs, preferably 150 to 3,000 nucleic acid basepairs, more preferably 150 to 1500 nucleic acid basepairs.

13. The method according to claim 1, wherein D is 10.

14. The method according to claim 1, wherein the number of replicate cultures prepared in step (e) is 2 to 500.

15. The method according to claim 3, wherein said mutant of a microorganism is obtained by homologous recombination.


SEQUENCE LISTING (14 sequences)

SEQ ID NO: 1 - DNA, 26 bp, Thermoanaerobacter sp.
  primer_bind (1)..(26): ldhup1F forward primer
ttccatatctgtaagtcccgctaaag

SEQ ID NO: 2 - DNA, 26 bp, Thermoanaerobacter sp.
  primer_bind (1)..(26): ldhup2 Reverse primer
attaatacaatagttttgacaaatcc

SEQ ID NO: 3 - DNA, 35 bp, Escherichia coli
  primer_bind (1)..(35): parM-ext-Fw forward primer for parM-ext sequence in E. coli plasmid R1/R100
ccccccgttaacatcaaactacagtggcaggaaag

SEQ ID NO: 4 - DNA, 41 bp, Escherichia coli
  primer_bind (1)..(41): parM-ext-re  reverse primer for parM-ext sequence in E. coli plasmid R1/R100
cccccctgcagcgttgcttcagatagttattatcttttctg

SEQ ID NO: 5 - DNA, 22 bp, Thermoanaerobacter sp.
  primer_bind (1)..(22): ldhdown3F forward primer
atataaaaagtcacagtgtgaa

SEQ ID NO: 6 - DNA, 22 bp, Thermoanaerobacter sp.
  primer_bind (1)..(22): ldhdown4R reverse primer
cacctattttgcactttttttc

SEQ ID NO: 7 - DNA, 21 bp, Escherichia coli
  primer_bind (1)..(21): parM-ext-Fw3  forward primer for parM-ext sequence in E. coli plasmid R1/R100
ggcaatacagcgacgttaatg

SEQ ID NO: 8 - DNA, 41 bp, Escherichia coli; identical to SEQ ID NO: 4
  primer_bind (1)..(41): parM-ext-Re  reverse primer for parM-ext sequence in E. coli plasmid R1/R100

SEQ ID NO: 9 - DNA, 510 bp, Escherichia coli
  gene (1)..(510): parM-ext fragment  from E. coli plasmid R1/R100
ggcaatacagcgacgttaatgtcgttgcagtgcatcacgccttactgaccagtggtctgccggtaagcgaagtggatattgtttgcacacttcctctgacagagtattacgacagaaataaccaacccaatacggaaaatattgagcgtaagaaagcaaacttccggaaaaaaattacattaaatggcggggatacattcacaataaaagatgtaaaagtcatgcctgaatctataccggcaggttatgaagttctacaagaactggatgagttagattctttattaattatagatctcgggggcaccacattagatatttctcaggtaatggggaaattatcggggatcagtaaaatgtacggagactcatctcttggtgtctctctggttacatctgcagtaaaagatgccctttctcttgcgagaacaaaaggaagtagctatcttgctgacgatataatcattcacagaaaagataataactatctgaagcaacgctgcagggggg

SEQ ID NO: 10 - DNA, 22 bp, Thermoanaerobacter sp.
  primer_bind (1)..(22): Primers LDH-out-Up  forward primer
gagctgctttaagtgtctcagg

SEQ ID NO: 11 - DNA, 24 bp, Thermoanaerobacter sp.
  primer_bind (1)..(24): LDH-out-Dn3  reverse primer
gaagtggatcctttataggccggt

SEQ ID NO: 12 - DNA, 23 bp, Pantoea ananatis
  primer_bind (1)..(23): budC_det_247_forward primer for BudC gene from Pantoea ananatis
aacgtsattgtgaataacgcmgg

SEQ ID NO: 13 - DNA, 20 bp, Pantoea ananatis
  primer_bind (1)..(20): budC_det_684_reverse primer or BudC gene from Pantoea ananatis
atcttccggctcnganaggc

SEQ ID NO: 14 - DNA, 341 bp, Pantoea ananatis
  gene (1)..(341): BudC gene encoding 2,3-butanediol dehydrogenase from Pantoea ananatis
aacgtcattgtgaataacgcaggtgtcgcgccctcaacgcccattgatgaaatcaccgaagaggtcatcaacaaggtttatgacgttaatgtcaaaggcgtgatctggggcatgcaggccgcaataaaagcgttcgcgtctgaaggtcatggcggaaaaattattaacgcctgttctcaggccgggcatgtcggcaatcccgagctggcagtttacagctcaagcaagtttgctgttcggggcctgacacaaactgccgcacgggatctggcaccggcggggatcaccgtgaatggcttttgtcctggtattgttaaaacaccgatgtgggcggagatcga